(19) United States
(12) Patent Application Publication
(10) Pub. No.: US 2010/0055803 A1
(43) Pub. Date: Mar. 4, 2010

(54) METHOD AND APPARATUS FOR DETECTING MOLECULES

(76) Inventor: Kwangyeol Lee, Namyangju-si (KR)

Correspondence Address: EDWARDS ANGELL PALMER & DODGE LLP, P.O. BOX 55874, BOSTON, MA, 02205, US

(21) Appl. No.: 12/202,028

(22) Filed: Aug. 29, 2008

(51) Int. Cl. G01N 33/553

(52) U.S. Cl. 436/526; 422/69

(57) ABSTRACT

A method and apparatus for detecting a target molecule in a sample are disclosed. The method optionally includes, but is not limited to, contacting the sample with a substrate having a metallic surface and receptors configured to bind to a target molecule, optionally in the presence of one or more metallic nanoparticles also including receptors configured to bind to a target molecule. The method optionally further includes dispersing a dye over the substrate; and applying a magnetic field to the substrate.

### BACKGROUND

Various methods for detecting the presence or concentration of molecules have been developed. For example, in biotechnology, a variety of methods for detecting DNA, RNA, or protein molecules are widely used. In certain detection methods, markers (for example, a fluorescent dye) are tagged to target molecules. Then, markers that are tagged to the target molecules are detected to indirectly determine the presence or concentration of the target molecules. Examples of such methods include flow cytometry, nucleic acid hybridization, DNA sequencing, nucleic acid amplification, immunoassays, histochemistry, and functional assays involving living cells fluorescence spectroscopy, and Raman spectroscopy.

In some instances, a concentration of target molecules in a sample may be low. In such instances, the methods described above may not be suitable for detecting the presence of the target molecules in the sample. In addition, tagging markers to target molecules may be time-consuming.

### SUMMARY

The systems, methods, and devices described herein each have several aspects, no single one of which is solely responsible for its desirable attributes.

An aspect by way of non-limiting example includes a method of detecting a target molecule in a sample. The method includes contacting the sample with a substrate including a surface formed of a first metallic material, wherein one or more first receptors configured to bind to the target molecule in the sample are coupled to the surface. The method also includes introducing one or more nanoparticles over the substrate. Each of the nanoparticles includes: a core, a coat covering at least a portion of the core, and one or more second receptors, wherein the core includes a magnetic material, wherein the coat is formed of a second metallic material, and wherein each of the second receptors is coupled to the nanoparticle, and is configured to bind to the target molecule. In some embodiments, the method further includes removing the sample from the substrate such that nanoparticles unlinked to the surface of the substrate are removed and the target molecule, if present, remains bound to the surface of the substrate through the first receptor. The method also includes dispersing a dye over the surface of the substrate; and applying a magnetic field to the substrate so as to bring the dye, the nanoparticle and the substrate together, wherein detection of the dye indicates the presence of the target molecule.

Another aspect by way of non-limiting example includes an apparatus for detecting a molecule. The apparatus includes a substrate that includes a metallic surface; one or more receptors attached to the metallic surface, wherein each of the one or more receptors is configured to bind to a target molecule; and a magnet configured to controllably apply a magnetic field to the substrate.

Yet another aspect by way of non-limiting example includes a nanoparticle. The nanoparticle includes a core, wherein the core includes a magnetic material. The nanoparticle also includes a coat covering at least a portion of the core, wherein the coat is formed of a metallic material. The nanoparticle further includes one or more receptors coupled to the nanoparticle, wherein the one or more receptors are configured to bind to a target. The magnetic material may include a paramagnetic or ferromagnetic material. The magnetic material may be dielectric. The magnetic material may include a metal oxide. The metal oxide may be at least one selected from the group consisting of iron oxides, maghemite, cobalt ferrite, magnesium ferrite, and manganese ferrite. The metallic material may be a noble metal. The noble metal may be at least one selected from the group consisting of gold (Au) and silver (Ag). The one or more receptors may be selected from the group consisting of an antibody, a ligand, an antigen and a nucleic acid.

Yet another aspect by way of non-limiting example includes a method of detecting a target molecule in a sample. The method includes contacting the sample with one or more nanoparticles. Each of said one or more nanoparticles includes a core that includes a magnetic material, a coat covering at least a portion of the core, and one or more first receptors coupled to the one or more nanoparticles, wherein the receptors are configured to bind to the target molecule. The method also includes contacting the sample/nanoparticles with a substrate. The substrate including a surface formed of a first metallic material, wherein one or more second receptors are configured to bind to the target molecule and are coupled to the surface. The method further includes removing nanoparticles that are unlinked to the substrate; contacting a dye with the surface of the substrate; and applying a magnetic field to the substrate so as to bring the dye, the nanoparticle and the substrate together, wherein detection of the dye indicates the presence of the target molecule.

The foregoing is a summary and thus contains, by necessity, simplifications, generalization, and omissions of detail; consequently, those skilled in the art will appreciate that the summary is illustrative only and is not intended to be in any way limiting. Other aspects, features, and advantages of the devices and/or processes and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE DRAWINGS

The foregoing and other features of the present disclosure will become more fully apparent from the following description and appended claims, taken in conjunction with the accompanying drawings. Understanding that these drawings depict illustrative embodiments and are not to be considered limiting, the disclosure will be described with additional specificity and detail through use of the accompanying drawings.

FIGS. 1A-1F show an illustrative embodiment of a method of detecting molecules.

FIGS. 2A-2F show another illustrative embodiment of a method of detecting molecules.

FIG. 3 is a schematic block diagram of an illustrative embodiment of a system for detecting molecules.

FIG. 4 is a schematic diagram of an illustrative embodiment of an apparatus for detecting molecules.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

The following detailed description is directed to certain specific embodiments. However, the embodiments can be varied in a multitude of different ways. As will be apparent from the following description, the embodiments may be implemented in or associated with a variety of devices and methods.

Embodiments relate to methods and materials that can be used to detect target substances. In particular, the methods and materials can be used to detect very small amounts of a target substance, that otherwise, might be undetectable due to the low signal emitted using other techniques. The methods and materials can detect small quantities of a target substance due to the enhancement of emission of dye molecules between a metal substrate and a metal nanoparticle.

In one aspect, a method of detecting a target molecule in a sample is provided. The method can include contacting the sample with a substrate including a surface formed of a first metallic material. One or more first receptors configured to bind to the target molecule in the sample are coupled to the surface. Examples of the first receptors include, but are not limited to, an antibody, an antigen, a ligand, and a nucleic acid.

The method also can include introducing one or more nanoparticles to the sample. Each of the nanoparticles can include a core, a coat covering at least a portion of the core, and one or more second receptors. The core includes a magnetic material. The coat is formed of a second metallic material. Each of the second receptors is coupled to the nanoparticle, and is configured to bind to the target molecule. Examples of the second receptors include, but are not limited to, an antibody, an antigen, a ligand, and a nucleic acid. The first and the second receptors may be the same and/or may be different.

The method further can include removing the sample from the substrate such that nanoparticles unlinked to the surface of the substrate are removed and the target molecule, if present, remains bound to the surface of the substrate through the first receptor. The method also can include dispersing a dye over the surface of the substrate; and applying a magnetic field to the substrate so as to bring the dye, the nanoparticle and the substrate together. Detection of the dye indicates the presence of the target molecule.

### Method of Detecting Molecules

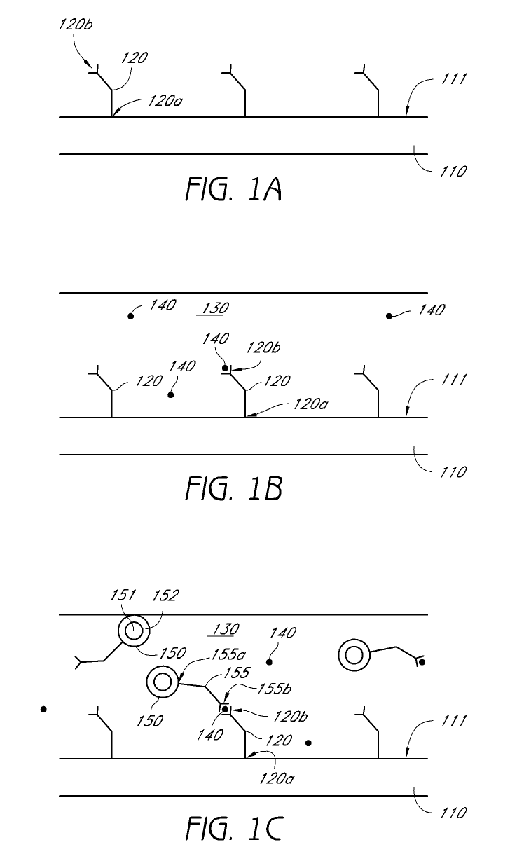

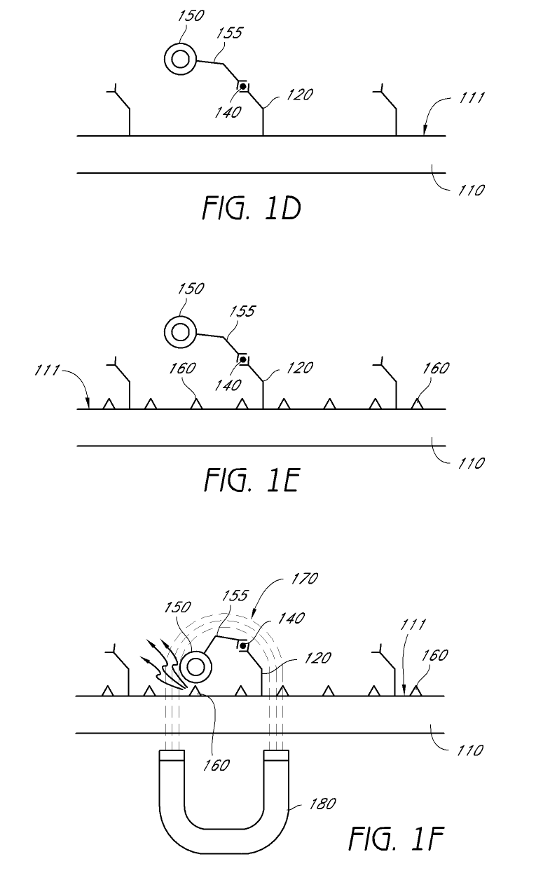

Referring to FIGS. 1A-1F, a method of detecting molecules according to one embodiment will be described below. First, a substrate 110 is provided, as shown in FIG. 1A. In one embodiment, the substrate may be formed of a material that can enhance the fluorescence of a fluorescent dye. In the context of this document, such a material can be referred to as a “fluorescence enhancing material.” Examples of fluorescence enhancing materials include, but are not limited to, noble metals, such as gold (Au) or silver (Ag).

When a fluorescent dye is sandwiched by two portions of a fluorescence enhancing material, the fluorescence of the dye can be significantly enhanced by a so-called “coupling effect.” A region created by two associated portions of a fluorescence enhancing material can be referred to as a hot spot. The details of the fluorescence enhancing material and hot spots are described in, for example, (1) Bek et al., “Fluorescence Enhancement in Hot Spots of AFM-Designed Gold Nanoparticles Sandwiches,” Nano Letters 2008, Vol. 8, No. 2, 485-490 (Jan. 4, 2008); (2) Zhang et al., “Metal-Enhanced Single-Molecule Fluorescence on Silver Particle Monomer and Dimer: Coupling Effect between Metal Particles,” Nano Letters 2007, Vol. 7, No. 7, 2101-2107 (Jun. 20, 2007); and (3) Nam et al., “Nanoparticle-Based Bio-Bar Codes for the Ultrasensitive Detection of Proteins,” Science, Vol. 301, 1884-1886 (Jul. 18, 2003). The disclosures of these articles are incorporated herein by reference in their entireties, including without limitation, for their disclosures related to metals and dyes that can be used, and for their methods of generating the enhanced fluorescence.

In another embodiment, the substrate 110 may include a plate formed of a non-fluorescence enhancing material, such as silicon or polydimethylsiloxane (PDMS). The non-fluorescence enhancing material can be any suitable solid material that does not react with a sample that is used during detecting molecules, as will be described below. In yet another embodiment, the substrate 110 may include a coating that prevents non-specific binding with molecules in samples. In such embodiments, the substrate 110 may further include a layer or film formed of a fluorescence enhancing material on a surface of the substrate.

The substrate 110 may also include a plurality of receptors 120 fixed to a surface 111 of the substrate 110, as shown in FIG. 1A. In the embodiment where the substrate 110 includes a plate and a layer of a fluorescence enhancing material, the receptors 120 may be fixed to the layer of the fluorescence enhancing material. The receptors 120 can have a first end 120a fixed to the surface 111 of the substrate 110, and a second end 120b configured to bind to one or more target molecules. The first end 120a of each of the receptors 120 can be attached to the surface 111 of the substrate 110. In an embodiment where the receptor 120 includes a thiol group, the receptor can be directly attached to the surface 111 using the thiol group (—SH). In other embodiments where the receptor 120 includes a group represented by —NH₂ or —COOH, but not a thiol group, a linker including a thiol group and a group represented by —COOH or —NH₂ can be used to attach the receptor to the surface 111.

Examples of receptors 120 include, but are not limited to, an antibody, a ligand, an antigen, and a nucleic acid. Examples of target molecules include, but are not limited to, a biomolecule, a cell, a nucleic acid, an antigen, an antibody, an aptamer, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, a liposome, and the like. In other embodiments, the target molecule can include any organic or inorganic molecule or a polymer that can be recognized by a receptor. In one embodiment, the density of the receptors 120 may be between about 2.5×10¹³/m² and about 2.5×10¹⁵/m², or optionally between about 10¹⁴/m² and about 10¹⁵/m². In certain embodiments, the density of the receptors 120 may be about 4×10¹⁴/m² (that is, about 1 receptor per 50 nm×50 nm), or about 10¹⁵/m², for example.

A sample 130 is provided over and/or to the substrate 110, as shown in FIG. 1B. The sample 130 can contain water, saline, a buffered solution or the like. The sample 130 may or may not contain target molecules. If the sample 130 contains target molecules 140, as shown in FIG. 1B, at least some of the target molecules 140 in the sample 130 can bind to the receptors 120. This step can be continued for a period of time that is sufficient to allow the receptors 120 to bind to the target molecules 140. The period of time can be between about 30 seconds and about 8 minutes, or optionally between about 1 minute and about 7 minutes. The period of time can be about 3 minutes or about 5 minutes, for example.

In some embodiments, nanoparticles 150 in a buffered solution are provided into the sample 130 over the substrate 110, as shown in FIG. 1C. The composition and PH of the buffered solution can vary widely, depending on the target molecules and their binding characteristics. The nanoparticles 150 can have a diameter between about 10 nm and about 300 nm, or optionally about between about 20 nm and about 200 nm. The diameter can be about 50 nm or about 100 nm, for example. In certain embodiments, this step can be carried out while applying a magnetic field to the substrate 110 such that the nanoparticles 150 are pulled close to the surface 111 of the substrate 110.

In an illustrative embodiment, the nanoparticles 150 include a core 151 and a coat 152 covering at least part of the core 151. In some embodiments, about 90 to about 100% of the surface of the core 151 is covered with the coat 152. For example, about 95% or about 100% of the core 151 can be covered with the coat 152. The core 151 may be formed of a magnetic (e.g., paramagnetic or ferromagnetic) material. In certain embodiments, the magnetic material is also dielectric. Examples of such materials include, but are not limited to, metal oxides, such as iron oxides (for example, magnetite (Fe₃O₄) or maghemite (γ-Fe₂O₃)), cobalt ferrite (CoFe₂O₄), magnesium ferrite (MgFe₂O₄), or manganese ferrite (MnFe₂O₄). The coat 152 can be formed of a fluorescence enhancing material, such as gold or silver. In one embodiment, the coat 152 can be formed of the same material as that of the substrate 110. In another embodiment, the coat 152 can be formed of a material different from that of the substrate 110. The coat 152 can have a thickness between about 1 nm and about 100 nm, optionally between about 5 nm and about 50 nm. The thickness of the coat 152 can be about 10 nm or about 50 nm, for example. Example methods of making the nanoparticles are disclosed in Xu et al., “Magnetic Core/Shell Fe3O4/Au and Fe3O4/Au/Ag Nanoparticles with Tunabale Plasmonic Properties, J.AM. CHEM. SOC, 2007, 129, 8698-8699, the disclosure of which is incorporated herein by reference in its entirety.

Each of the nanoparticles 150 can also include at least one receptor 155. The receptor 155 may include a first end 155a fixed to the coat 152 and/or the core 151, and a second end configured to bind to one or more of the target molecules 140. In one embodiment, the first end 155a of the receptor 155 can be attached to the coat 152 in the manner described above with respect to the receptors 120. Any entity or material can be used as a receptor to capture, bind to, secure, or otherwise recognize and receive a target material. Examples of receptors 155 include, but are not limited to, an antibody, a ligand, an antigen, and a nucleic acid. For example, the receptor can be an antibody that binds to a specific antigen or visa-versa, a lectin that can bind a carbohydrate or visa versa, a nucleic acid-nucleic acid, biotin binding to avidin or the reverse, etc. The receptor 155 of each of the nanoparticles 150 can be a material the same as or different from that of the receptor 120 fixed to the substrate 110. Other examples of entities or categories of materials that can be used as “receptors” include a protein, a peptide, a polypeptide, Fab fragment, a drug, a small molecule chemical, a cell, a metabolite, an enzyme, and analyte, an anti-ligand, and a marker. Also, the receptor in some aspects need not be limited to single binding partners but may include the interactions of multiple binding partners. It should be understood that the receptor categories and entities listed above are not necessarily mutually exclusive. In fact, in some cases certain entities may fall under several separate categories and several categories can be overlapping.

A “ligand” may be generally defined as any molecule for which there exists another molecule (i.e. an anti-ligand) which specifically or non-specifically binds to said ligand, owing to recognition of some portion of said ligand. An “anti-ligand” can be defined as any molecule that specifically or non-specifically binds to another molecule (ligand). For example, an anti-ligand can be an antibody and the ligand a molecule such as an antigen which binds specifically to the antibody. A ligand may also consist of cells, cell membranes, organelles and synthetic analogues thereof As used herein, the term “nucleic acid” refers to a deoxyribonucleotide or ribonucleotide polymer in either single- or double-stranded form, and encompasses analogs of natural nucleotides that can function in a similar manner as naturally occurring nucleotides.

A nucleic acid may act as a receptor, for example, by binding to another nucleic acid of the same or different type (i.e. deoxyribonucleotide, ribonucleotide, or analog of natural nucleotide) that has a complementary or largely complementary sequence. A nucleic acid may also act as a receptor by binding to DNA or RNA binding proteins, such as ribosomes, polymerases, histones, gyrases, exonucleases, etc.

As used herein, the terms “polypeptide,” “peptide” and “protein” can be used interchangeably to refer to a polymer of amino acid residues. The terms also can apply to amino acid polymers in which one or more amino acid residue is a modified and/or an artificial chemical analogue of a corresponding naturally occurring amino acid, as well as to naturally occurring amino acid polymers. Polypeptides and proteins can include receptors, antibodies, antibody fragments, ligands, signaling molecules, enzymes, substrates, antigens, and epitopes, for example. Polypeptides, peptides and proteins may act as receptors by binding specifically or non-specifically with a binding partner for which it has an affinity.

As used herein, the term “antibody” can mean an immunoglobulin molecule or a fragment of an immunoglobulin molecule having the ability to specifically bind to a particular antigen. The term “antibody” can mean not only full-length antibody molecules but also fragments of antibody molecules retaining antigen binding ability. In particular, as used herein, the term “antibody” includes not only full-length immunoglobulin molecules, but also antigen binding active fragments such as active fragments F(ab′)₂, Fab, Fv, and Fd. Antibodies can act as receptors by binding to antigens and also can be targets where an antigen is acting as the receptor.

The term “antigen” can include a molecule that contains one or more epitopes capable of stimulating a host's immune system to make a cellular antigen-specific immune response when the antigen is presented, or a humoral antibody response. An antigen may be capable of eliciting a cellular and/or humoral response by itself or when present in combination with another molecule. An “epitope” is that portion of an antigenic molecule or antigenic complex that determines its immunological specificity. An epitope is within the scope of the present definition of antigen. Antigens may act as receptors by binding to antibodies or to antibody fragments, for example.

As used herein, the term “enzyme,” can refer to a protein which acts as a catalyst to reduce the activation energy of a chemical reaction in other compounds or “substrates,” but is not a final product in the reaction. An enzyme may act as a receptor by binding to its substrate, for example, or a substrate can act as a receptor by binding to an enzyme.

As used herein, the term “analyte” can refer to a molecular entity whose presence, structure, binding ability, etc., is being detected or analyzed. Suitable analytes for use as receptors include, but are not limited to, antibodies, antigens, nucleic acids (e.g., natural or synthetic DNA, RNA, gDNA, cDNA, mRNA, tRNA), lectins, sugars, glycoproteins, receptors and their cognate ligand (e.g. growth factors and their associated receptors, cytokines and their associated receptors, signaling molecules and their receptors), small molecules such as existing pharmaceuticals and drug candidates (either from natural products or synthetic analogues developed and stored in combinatorial libraries), metabolites, drugs of abuse and their metabolic by-products, co-factors such as vitamins and other naturally occurring and synthetic compounds, oxygen and other gases found in physiologic fluids, cells, phages, viruses, cellular constituents cell membranes and associated structures, other natural products found in plant and animal sources, and other partially or completely synthetic products. Analytes can be targets of receptors or in some cases can act as receptors. For example, analytes such as small molecules, drugs, metabolites, sugars, etc. can bind receptors, channels, ligands, for example.

If the sample 130 contains target molecules 140, the receptors 155 of the nanoparticles 150 can bind to the target molecules 140. At least some of the receptors 155 of the nanoparticles 150 can bind to target molecules 140 that have been bound to the receptors 120 fixed to the substrate 110, as shown in FIG. 1C. In this manner, certain nanoparticles 150 can be linked to the substrate 110 via the target molecules 140. This step can be continued for a period of time that is sufficient to allow the receptors 155 to bind to the target molecules 140. The period of time can be between about 30 seconds and about 8 minutes, optionally between about 1 minute and about 7 minutes. The period of time can be about 5 minutes, for example.

The sample 130 is removed from over the substrate 110. In one embodiment, the top surface of the substrate 110 is further washed so as to remove unlinked nanoparticles while keeping the linked nanoparticles over the substrate 110. In one embodiment, the top surface of the substrate 110 may be repeatedly washed with a buffer solution having a pH of about 5 to 9. As a result, only linked nanoparticles 150 remain over the substrate 110, as shown in FIG. 1D.

In some embodiments, a solution containing a dye is applied or provided to (e.g., spread or sprayed or contacted) and dried on the surface 111 of the substrate 110, leaving dye portions 160 dispersed, as shown in FIG. 1E, for example. The dye can be dried or used without being dried. In certain embodiments, at least one of the dye portions 160 can be formed by a single dye molecule. In some embodiments, this step can be performed at a point before removing the sample 130 from contact with the substrate 110.

In one embodiment, the dye can be a fluorescent dye that can be used for fluorescence spectroscopy. Examples of fluorescent dyes include, but are not limited to, ethidium bromide, SYBR Green, fluorescein isothiocyanate (FITC), DyLight Fluors (available from Thermo Fisher Scientific, Waltham, Mass.), green fluorescent protein (GFP), or the like. A skilled artisan will appreciate that any other suitable fluorescent dyes can also be used for the embodiment. The solution can have a relatively low concentration of the dye such that the fluorescence emitted by the dye portions 160 is undetectable by bare eyes. In one embodiment, the density of the dye portions 160 can be about 10¹⁶ to about 5×10¹⁸ molecules/m² when the dye molecule has a size of about 1 nm. In other embodiments, the density of the dye portions 160 can be about 10¹⁶ to about 10¹⁸ molecules/cm², optionally about 5×10¹⁷ molecules/m², for example.

In some embodiments, a magnetic field 170 is applied to the substrate 110 by a magnet 180. The magnetic field 170 serves to pull the nanoparticles 150 to the surface 111 of the substrate 110, as shown in FIG. 1F. The magnetic field may have a magnitude between about 0.001 T and about 100 T, optionally between about 0.01 T and about 10 T, or optionally about 5 T, for example. In other embodiments, the magnetic field 170 may be generated by an electromagnet.

As illustrated in FIG. 1F, if there is a target molecule 140 linking a nanoparticle 150 to the substrate 110, a hot spot can be created by pulling the nanoparticle 150 close to the substrate 110. Thus, the fluorescence of the dye portion 160 sandwiched by the nanoparticle 150 and the substrate 110 can be enhanced by, for example, about 1.1 to about 100 times. In one embodiment, the enhanced fluorescence may be detected by direct visualization (for example, observation by bare eyes), although the fluorescence of the dye portion 160 may not be detected by direct visualization when there is no application of the magnetic field. In other embodiments, although the enhanced fluorescence may not be detected by bare eyes, a fluorescence detector can be used to sense the enhanced fluorescence.

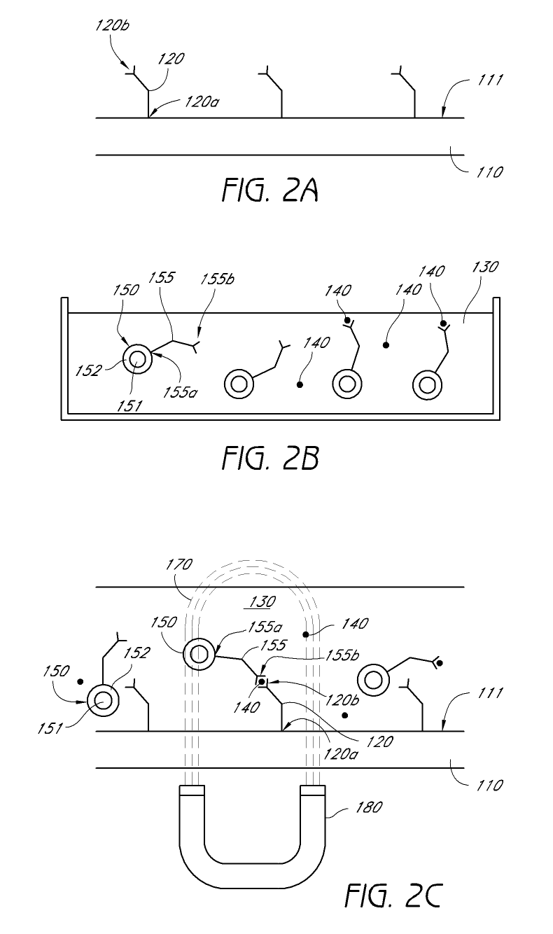

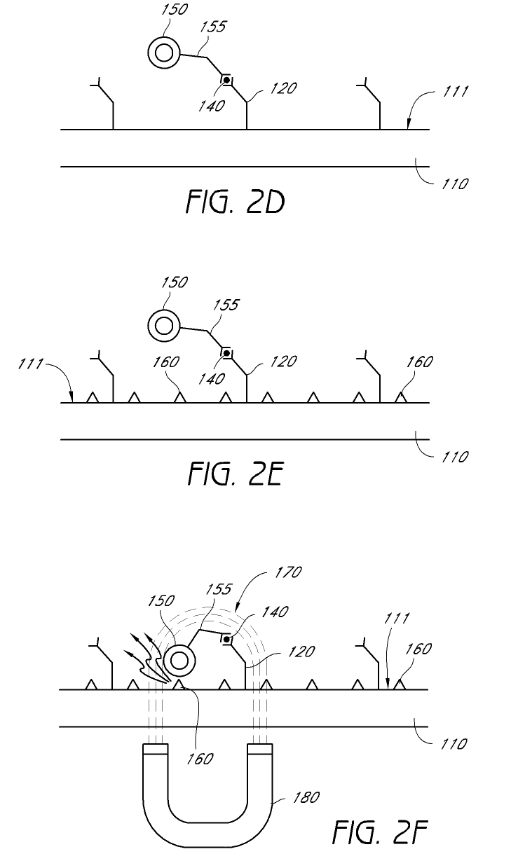

Referring to FIGS. 2A-2F, a method of detecting molecules according to another embodiment will be described below. First, a substrate 110 is provided, as shown in FIG. 2A. The substrate 110 includes a surface 111 and a plurality of receptors 120 attached to the surface 111. The receptors 120 can have a first end 120a fixed to the surface 111 of the substrate 110, and a second end 120b configured to bind to one or more target molecules. The other details of the substrate 110 can be as described above in connection with FIG. 1A.

A sample 130 is mixed with nanoparticles 150, as shown in FIG. 2B. The sample 130 can contain water, saline, a buffered solution or the like. The nanoparticles 150 can have a diameter between about 10 nm and about 50 nm. In the illustrated embodiment, each of the nanoparticles 150 includes a core 151 and a coat 152 covering at least part of the core 151. The details of the nanoparticles 150 can be as described above in connection with FIG. 1C.

Each of the nanoparticles 150 can also include at least one receptor 155. The receptor 155 may include a first end 155a fixed to the surface of the coat 152 and/or the core 151, and a second end configured to bind to one or more of the target molecules 140. The receptor 155 of each of the nanoparticles 150 can be a material the same as or different from that of the receptor 120 fixed to the substrate 110.

The sample 130 may or may not contain target molecules. If the sample 130 contains target molecules 140, as shown in FIG. 2B, at least some of the target molecules 140 in the sample 130 can bind to the receptors 155 of the nanoparticles 150. This step can be performed with or without stirring or agitation for a period of time that is sufficient to allow the target molecules 140 to bind to the receptors 155. In one embodiment, the period of time can be between about 30 seconds and about 10 minutes, optionally between about 30 seconds and 5 minutes, for example. The period of time can be about 3 minutes or about 5 minutes, for example. This step can be performed at a temperature between about 5° C. and about 70° C., optionally between about 5° C. and about 40° C. The temperature can be, for example, 5° C. or about 25° C.

A mixture resulting from the step shown in FIG. 2B is provided to the substrate 110, as shown in FIG. 2C. In some embodiments, the mixing may be performed in the presence of the substrate. In the illustrated embodiment, a magnetic field 170 is applied by a magnet 180 such that the nanoparticles 150 in the sample 130 are attracted toward the surface 111 of the substrate 110. This facilitates the binding of the receptors 120 of the substrate 110 to target molecules that have been bound to the nanoparticles 150.

If the sample 130 contains target molecules 140, the receptors 120 of the substrate 110 can bind to the target molecules 140. At least some of the receptors 120 of the substrate 110 can bind to target molecules 140 that have been bound to the receptors 155 of the nanoparticles 150, as shown in FIG. 2C. In this manner, certain nanoparticles 150 can be linked to the substrate 110 via the target molecules 140. This step can be continued for a period of time that is sufficient to allow the receptors 120 to bind to the target molecules 140. The period of time can be between about 2 minutes and about 8 minutes, or between about 3 minutes and about 6 minutes. The period of time can be optionally about 5 minutes, for example.

The sample 130 is removed from the substrate 110 in the absence of a magnetic field. In one embodiment, the surface of the substrate 110 is further washed so as to remove unlinked nanoparticles while keeping the linked nanoparticles over the substrate 110. As a result, only the linked nanoparticles 150 remain over the substrate 110, as shown in FIG. 2D.

A solution containing a dye is applied, spread and optionally dried on the surface 111 of the substrate 110, leaving dye portions 160 sparsely distributed, as shown in FIG. 2E. In certain embodiments, at least one of the dye portions 160 can be formed by a single dye molecule. In other embodiments, this step can be performed before removing the sample 130 from over the substrate 110. The details of this step can be as described above in connection with FIG. 1E.

A magnetic field 170 is optionally re-applied to the substrate 110 by the magnet 180. The magnetic field 170 serves to pull the nanoparticles 150 to the surface 111 of the substrate 110, as shown in FIG. 2F. In other embodiments, the magnetic field 170 may be generated by an electromagnet.

As illustrated in FIG. 1F, if there is a target molecule 140 linking a nanoparticle 150 to the substrate 110, a hot spot can be created by pulling the nanoparticle 150 close to the substrate 110. Such a hot spot can be created when metallic surfaces are positioned close to each other with a distance of, for example, about 5 nm or less. The details of the hot spots are described in, for example, Bek et al., “Fluorescence Enhancement in Hot Spots of AFM-Designed Gold Nanoparticles Sandwiches,” Nano Letters 2008, Vol. 8, No. 2, 485-490 (Jan. 4, 2008). Thus, the fluorescence of the dye portion 160 sandwiched by the nanoparticle 150 and the substrate 110 can be enhanced. In one embodiment, the enhanced fluorescence may be detected by bare eyes, although the fluorescence of the dye portion 160 may not be detected by bare eyes when there is no application of the magnetic field. In other embodiments, although the enhanced fluorescence may not be detected by bare eyes, a fluorescence detector can be used to sense the enhanced fluorescence.

In another embodiment, the embodiments described above can be adapted for Raman spectroscopy. For example, the embodiment can be adapted for Surface-Enhanced Raman Spectroscopy (SERS). The details of SERS is disclosed, e.g., in Talley et al., “Surface-Enhanced Raman Scattering from Individual Au Nanoparticles and Nanoparticle Dimer Substrates,” Nano Letters 2005, Vol. 5, No. 8, 1569-1574 (Jun. 28, 2005), the disclosure of which is incorporated herein by reference in its entirety.

In one embodiment, a substrate is provided, as described above in connection with FIG. 1A. A sample is provided over the substrate, as described above in connection with FIG. 1B. Nanoparticles are provided to the sample over the substrate, as described above in connection with FIG. 1C. The unbound sample is removed from over the substrate.

A solution containing Raman active molecules is spread and dried on the surface of the substrate, leaving the Raman active molecules in a sparsely distributed manner, as described above in connection with FIG. 1E. Examples of Raman active molecules can include, but are not limited to, TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N, N-diethyl-4-(5′-azobenzotriazolyl)-phenylamine and aminoacridine. These and other Raman-active molecules can be obtained from commercial sources (e.g., Molecular Probes, Eugene, Oreg.).

A magnetic field is provided to the substrate, as described above in connection with FIG. 1F. As illustrated in FIG. 1F, if there is a target molecule linking a nanoparticle to the substrate, a hot spot can be created by pulling the nanoparticle close to the substrate. Then, a laser for Raman scattering is illuminated onto the substrate. The dye portion sandwiched by the nanoparticle and the substrate can provide enhanced Raman scattering.

In another embodiment for Raman spectroscopy, a substrate is provided, as described above in connection with FIG. 2A. A sample is mixed with nanoparticles, as described above in connection with FIG. 2B. The sample is provided over the substrate, as described above in connection with FIG. 2C. The sample is removed from over the substrate, as described above in connection with FIG. 2D.

A solution containing Raman active molecules is spread and dried on the surface of the substrate, leaving the Raman active molecules in a sparsely distributed manner, as described above in connection with FIG. 2E. A magnetic field is provided to the substrate, as described above in connection with FIG. 2F. As illustrated in FIG. 2F, if there is a target molecule linking a nanoparticle to the substrate, a hot spot can be created by pulling the nanoparticle close to the substrate. Then, laser is illuminated on the substrate. The Raman active molecules sandwiched by the nanoparticle and the substrate can provide enhanced Raman scattering.

### System and Apparatus for Detecting Molecules

Referring to FIG. 3, one embodiment of a system for detecting molecules will be described below. The illustrated system 300 includes a molecule detector 310, a reservoir 320, a first conduit 330, and a second conduit 340. The molecule detector 310 can also be referred to as a “molecular sensor.” The molecule detector 310 is in fluid communication with the reservoir 320 via a first conduit 320. The molecule detector 310 may discard a fluid through a second conduit 340. The conduits 330, 340 can be equipped with valves (not shown). The valves may be controlled, including e.g., manually, automatically, remotely, and the like.

The reservoir 320 may contain a sample and provide the sample to the detector 310 through the first conduit 330. The detector 310 can be configured to detect target molecules according to the methods described above in connection with FIGS. 1A-1F or FIGS. 1A-1F. In the illustrated embodiment, the detector 310 is a fluorescence detector. In other embodiments, the detector 310 can also be adapted for Raman spectroscopy. After the sample is read by the detector 310, the sample can be discarded through the second conduit 340.

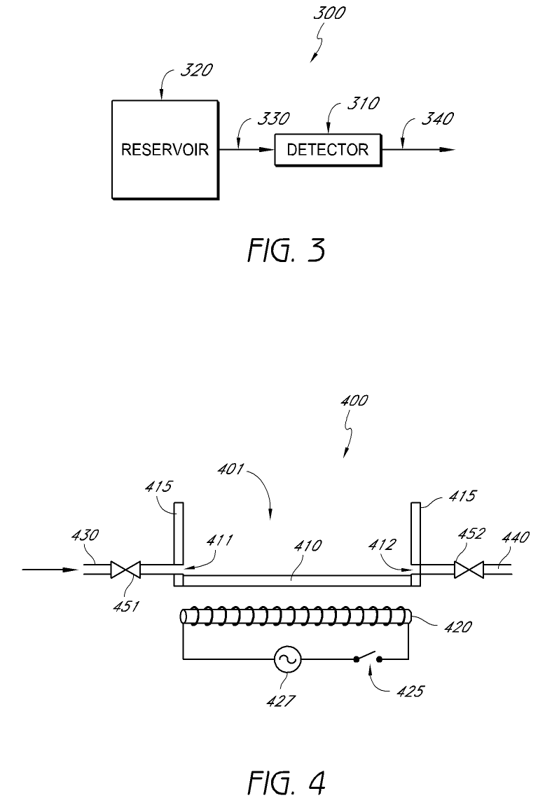

Referring to FIG. 4, one embodiment of an apparatus for detecting molecules will be described below. The apparatus can serve as part of a detector 310 in the system 300 of FIG. 3. The illustrated apparatus 400 includes a substrate 410, a sidewall 415, an electromagnet 420, a first conduit 430, a second conduit 440, a first valve 451, and a second valve 452. The substrate 410 and the sidewall 415 may form a container 401 for a sample during the detection of molecules. The details of the substrate 410 can be as described above in connection with FIG. 1A.

The sidewall 410 includes an inlet 411 and an outlet 412. The inlet 411 is in fluid communication with the first conduit 430. The outlet 412 is in fluid communication with the second conduit 440. The first conduit 430 is equipped with the first valve 451 for opening, closing, or controlling a flow there through. The second conduit 440 is equipped with the second valve 452 for opening, closing, or controlling a flow there through.

The electromagnet 420 is positioned, oriented, and configured to apply a magnetic field substantially across the substrate 410 such that nanoparticles can be attracted to the substrate 420 in the manner described above in connection with FIG. 1F. The electromagnet 420 can be equipped with a switch 425 that can selectively connect the electromagnet 420 to a power source 427.

In one embodiment, during operation, a sample is provided over the substrate 410 through the first conduit 430 and the inlet 411, as described above in connection with FIG. 1B. Subsequently, nanoparticles are provided into the sample over the substrate 410, as described above in connection with FIG. 1C. The nanoparticle may be provided through another conduit (not shown). The sample is removed from over the substrate 410 through the outlet 412 and the second conduit 440.

Next, a solution containing a dye is provided through the top of the container 401, and is spread and dried on the surface of the substrate 410, leaving dye portions in a sparsely distributed manner, as described above in connection with FIG. 1E. In this embodiment, the dye can be a fluorescent dye. In other embodiments, Raman active molecules can be used instead of the dye.

Subsequently, a magnetic field is provided to the substrate by the electromagnet 420, as described above in connection with FIGS. 1F and 2F. As illustrated in FIGS. 1F and 2F, if there is a target molecule linking a nanoparticle to the substrate 410, a hot spot can be created by pulling the nanoparticle close to the substrate 410. Thus, the dye portion sandwiched by the nanoparticle and the substrate can provide enhanced fluorescence or Raman scattering. The fluorescence or Raman scattering can be detected by a spectrometer. A skilled artisan will appreciate that the spectrometer can be positioned at a location suitable for detecting the fluorescence or Raman scattering. In the illustrated embodiment, the steps described above may be automatically performed by the control of a computer or microprocessor. In another embodiment, the apparatus can operate according to the method described above in connection with FIGS. 2A-2F. A skilled artisan will appreciate that the configuration and operation of the apparatus can vary widely, depending on the applications.

In another embodiment, a kit for detection of molecules can be provided. The kit may include an apparatus for detecting a molecule. The apparatus includes a substrate that includes a metallic surface; one or more receptors attached to the metallic surface; and a magnet configured to controllably apply a magnetic field to the substrate. Each of the one or more receptors is configured to bind to a target molecule. The details of the substrate can be as described above with respect to FIG. 1A. The details of the one or more receptors can be as described above with respect to the receptors of 120 of FIG. 1A. The details of the magnet can be as described above with respect to the magnet 180 of FIG. 1F.

The kit may also include one or more nanoparticles that include a core; a coat covering at least a portion of the core; and one or more receptors coupled to the nanoparticle. The core includes a magnetic material. The coat is formed of a metallic material. The one or more receptors are configured to bind to a target molecule. The details of the nanoparticles can be as described above with respect to the nanoparticles 150 of FIG. 1C. In one embodiment, the kit may also include a dye (for example, a fluorescent dye) or Raman active molecules.

The embodiments above are described in the context of detection of molecules by fluorescence or Raman scattering. A skilled artisan will, however, appreciate that the embodiments can be adapted for any type of spectroscopy (for example, those employing Fluorescence resonance energy transfer (FRET) or fluorescence emitting quantum dots) that employs emission that can be enhanced by coupling effect in a hot spot.

The methods and apparatus described above can be adapted for detection of molecules at a relatively low concentration in various applications. For example, the methods can be used as a biosensor in chemical, biological, or pharmaceutical research, or disease diagnostics.

In at least some of the aforesaid embodiments, any element used in an embodiment can interchangeably be used in another embodiment unless such a replacement is not feasible. It will be appreciated that the steps of the methods described above can be combined, divided, or omitted or that additional steps can be added. It will also be appreciated by those skilled in the art that various other omissions, additions and modifications may be made to the methods and structures described above without departing from the scope of the embodiments.

For purposes of this disclosure, certain aspects, advantages, and novel features of the embodiments are described herein. It is to be understood that not necessarily all such advantages may be achieved in accordance with any particular embodiment. Thus, for example, those skilled in the art will recognize that some embodiments may be embodied or carried out in a manner that achieves one advantage or group of advantages as taught herein without necessarily achieving other advantages as may be taught or suggested herein.

The herein described subject matter sometimes illustrates different components contained within, or connected with, different other components. It is to be understood that such depicted architectures are merely illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of detecting a target molecule, the method comprising:
contacting a sample with a substrate including a surface formed of a first metallic material, wherein one or more first receptors configured to bind to the target molecule are coupled to the surface;
introducing one or more nanoparticles over the substrate, each of the nanoparticles including: a core, a coat covering at least a portion of the core, and one or more second receptors, wherein the core includes a magnetic material, wherein the coat is formed of a second metallic material, and wherein the second receptors are coupled to the nanoparticle, and are configured to bind to the target molecule;
removing the sample from the substrate such that nanoparticles unlinked to the surface of the substrate are removed and the target molecule, if present, remains bound to the surface of the substrate through the first receptor;
dispersing a dye over the surface of the substrate; and
applying a magnetic field to the substrate to bring the dye, the one or more bound nanoparticles and the substrate into association, wherein the association of the nanoparticles and the substrate in the presence of the dye indicates the presence of the target molecule.

2. The method of claim 1, wherein the first metallic material is the same as the second metallic material.

3. The method of claim 1, wherein at least one of the first or second metallic material is a noble metal.

4. The method of claim 3, wherein the noble metal is at least one selected from the group consisting of gold (Au) and silver (Ag).

5. The method of claim 1, wherein the target molecule comprises one selected from the group consisting of a DNA molecule, an RNA molecule, an oligonucleotide, and a protein.

6. The method of claim 1, wherein the first receptors are identical to the second receptors.

7. The method of claim 1, wherein the first and second receptors are selected from the group consisting of an antibody, a ligand, an antigen and a nucleic acid.

8. The method of claim 1, wherein the one or more second receptors are coupled to the core and/or the coat of the nanoparticle.

9. The method of claim 1, wherein the magnetic material comprises a paramagnetic or ferromagnetic material.

10. The method of claim 9, wherein the magnetic material is dielectric.

11. The method of claim 10, wherein the magnetic material comprises a metal oxide.

12. The method of claim 11, wherein the metal oxide comprises at least one selected from the group consisting of iron oxides, maghemite, cobalt ferrite, magnesium ferrite, and manganese ferrite.

13. The method of claim 1, wherein applying the magnetic field comprises using a magnet and/or an electromagnet.

14. The method of claim 1, further comprising:
preparing a mixture of the sample and the nanoparticles and contacting the mixture with the substrate.

15. The method of claim 14, wherein preparing the mixture comprises stirring the mixture.

16. The method of claim 14, wherein applying the magnetic field comprises applying the magnetic field while contacting the mixture with the substrate.

17. The method of claim 1, wherein applying the magnetic field comprises applying the magnetic field while introducing the nanoparticles over the substrate.

18. The method of claim 1, wherein removing the sample does not include applying a magnetic field to the substrate.

19. The method of claim 1, wherein the dye is a fluorescent material, and wherein the method further comprises detecting the fluorescence of the dye while applying the magnetic field.

20. The method of claim 1, wherein the dye comprises a Raman active molecule, and wherein the method further comprises detecting the Raman-scattering of the Raman active molecule while applying the magnetic field.

21. The method of claim 1, wherein the dye comprises one or more selected from the group consisting of ethidium bromide, SYBR Green, fluorescein isothiocyanate (FITC), DyLight Fluors, green fluorescent protein (GFP), TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N, N-diethyl-4-(5′-azobenzotriazolyl)-phenylamine and aminoacridine.

22. An apparatus for detecting a molecule, comprising:
a substrate that includes a metallic surface;
one or more receptors attached to the metallic surface, wherein the one or more receptors is configured to bind to a target molecule; and
a magnet configured to controllably apply a magnetic field to the substrate.

23. The apparatus of claim 22, further comprising a sidewall that forms a container together with the substrate.

24. The apparatus of claim 23, wherein the sidewall includes at least one inlet configured to provide a fluid there through to the substrate, and at least one outlet configured to remove the fluid from the substrate.

25. The apparatus of claim 22, wherein the metallic surface is formed of a noble metal.

26. The apparatus of claim 25, wherein the noble metal is selected from the group consisting of gold (Au) and silver (Ag).

27. The apparatus of claim 22, wherein the target molecule comprises one selected from the group consisting of a DNA molecule, an RNA molecule, an oligonucleotide, and a protein.

28. The apparatus of claim 22, further comprising a spectrometer configured to detect fluorescence or Raman scattering.

29. The apparatus of claim 22, wherein the one or more receptors is selected from the group consisting of an antibody, a ligand, an antigen, and a nucleic acid.

30. The apparatus of claim 22, wherein the magnet is an electromagnet.

31. The apparatus of claim 22, wherein the magnet controllably applies the magnetic field by being distanced from the surface, by being turned on or off, or by being blocked.

32. A kit comprising:
the apparatus of claim 22; and
one or more nanoparticles that include:
a core, wherein the core includes a magnetic material,
a coat covering at least a portion of the core, wherein the coat is formed of a metallic material, and
one or more receptors coupled to the nanoparticle, wherein the one or more receptors are configured to bind to a target molecule.

33. The kit of claim 32, further comprising a dye.

34. The kit of claim 33, wherein the dye comprises one or more selected from the group consisting of ethidium bromide, SYBR Green, fluorescein isothiocyanate (FITC), DyLight Fluors, green fluorescent protein (GFP), TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N,N-diethyl-4-(5′-azobenzotriazolyl)-phenylamine and aminoacridine.

35. A method of detecting a target molecule in a sample, the method comprising:
contacting the sample with one or more nanoparticles, each of said one or more nanoparticles including a core that includes a magnetic material, a coat covering at least a portion of the core, and one or more first receptors coupled to the one or more nanoparticles, wherein the receptors are configured to bind to the target molecule;
providing the sample contacted with the one or more nanoparticles to a substrate, the substrate including a surface formed of a first metallic material, and wherein one or more second receptors are configured to bind to the target molecule and are coupled to the surface;
removing nanoparticles that are unlinked to the substrate;
providing a dye to the surface of the substrate; and
applying a magnetic field to the substrate so as to bring the dye, the nanoparticle and the substrate together, wherein detection of the dye indicates the presence of the target molecule.

36. The method of claim 35, further comprising detecting a light characteristic.

37. The method of claim 36, wherein the light characteristic is a coupling effect due to the sandwiching of the dye with the nanoparticle and the metallic surface of the substrate.
